(19) United States
(12) Patent Application Publication
(10) Pub. No.: US 2012/0244197 A1
(43) Pub. Date: Sep. 27, 2012

(54) Film-Like Pharmaceutical Dosage Forms

(75) Inventors: Dejan Djuric, Mannheim (DE); Karl Kolter, Limburgerhof (DE); Michael Gerrit Herting, Mannheim (DE)

(73) Assignee: BASF SE, Ludwigshafen (DE)

(21) Appl. No.: 13/511,759

(22) PCT Filed: Nov. 12, 2010
(86) PCT No.: PCT/EP10/67384

§ 371 Date: May 24, 2012

Related U.S. Application Data
(60) Provisional application No. 61/264,259, filed on Nov. 25, 2009.

(30) Foreign Application Priority Data
Nov. 24, 2009 (EP) 09176929.9

(51) Int. Cl. A61K 47/34; A61K 47/38; B29C 47/00; A61K 9/70

(52) U.S. Cl. 424/400; 514/772.1; 514/772.2; 264/177.1

(57) ABSTRACT

Film-like pharmaceutical dosage forms, comprising, as film formers, amphiphilic copolymers and one or more active substances.

The present invention relates to film-like pharmaceutical dosage forms based on amphiphilic copolymers as film formers.

The invention describes physiologically tolerated active substance-containing films for use in humans or animals.

The films can be used as plaster inlays and wound dressings and in particular also for oral administration.

In the case of film-like dosage forms which can be administered orally, also referred to as “oral strips”, the higher permeability of the buccal mucosa in comparison with the skin can be utilized. Because of this and also because of possible circumventing of the first pass effects, it is also possible to realize higher absorption rates or higher bioavailabilities.

The major advantage of oral films in pharmacy is that they can readily be used both in pediatrics and in geriatrics. They can be readily metered and can generally be taken readily without additional liquid. Because of this, this novel drug form is particularly suitable for therapy in the case of difficulties in swallowing, nausea, attacks of dizziness and emotional disturbances.

Typically, polymers are used for producing films. As further additives, further polymers, active substances, plasticizers or aromas may also be added. Melt extrusion or the evaporation method are known and established as production methods according to the prior art. The following may be mentioned as examples here: hydroxypropylmethylcellulose (hypromellose), hydroxypropylcellulose, starch and modified starch, pullulan, pectin, gelatin and carboxymethylcellulose (Dixit and Puthli, Journal of Controlled Release 139 (2009) 94-107).

The problem in the preparation of such film-like dosage forms consists however in the choice of a suitable base material for the films. This base material, which is intended to form the matrix of the film, must be readily processable to give films; furthermore, the active substance must be capable of being readily incorporated and, for reasons relating to drug safety, the film must have a high mechanical strength in combination with a good release profile for the active substance.

A major disadvantage of the films known to date is that they have too low a dissolving power for active substances and the active substance is therefore present in crystalline form, with the result that it has poor bioavailability. Moreover, a grainy sensation in the mouth may be produced thereby. Two-phase systems generally entail the problem of homogeneity and of uniformity of content. The flexibility, too, is frequently low, with the result that they can easily break or tear. The polymers known to date tend to be hydrophilic and, owing to their high glass transition temperature and high viscosity, are scarcely extrudable, extrudable only at high temperature or difficult to produce from solutions by knife coating. In the knife coating process, inhomogeneities and air inclusions frequently occur.

Amphiphilic copolymers, such as graft polymers, obtained by free radical polymerization of vinyl acetate and N-vinyllactams in the presence of a polyether, are known per se.

WO 2007/051743 discloses the use of water-soluble or water-dispersible copolymers of N-vinyllactam, vinyl acetate and polyethers, as solubilizers for pharmaceutical, cosmetic, food, agrotechnical or other technical applications. It is stated very generally therein that the corresponding graft polymers can also be processed in the melt with the active substances.

WO 2009/013202 discloses that such graft polymers of N-vinyllactam, vinyl acetate and polyethers are melted in an extruder and mixed with pulverulent or liquid active substances and can be processed to give tablets.

It was an object of the present invention to provide improved film-like dosage forms which have advantages over the prior art with respect to production and handling of the films, mechanical strength and release behavior.

Accordingly, film-like dosage forms comprising, as a film former, an amphiphilic copolymer and one or more active substances and, if appropriate, further pharmaceutical excipients were found.

The film-like dosages forms may comprise the amphiphilic copolymers in amounts of from 1 to 100% by weight, preferably from 10 to 90% by weight, particularly preferably from 40 to 70% by weight, based on the total amount of pharmaceutical excipients.

The content of active substance depends on its effective dose per dosage form.

Suitable amphiphilic copolymers are in particular copolymers of polyethers, N-vinyl monomers and further vinyl monomers.

Copolymers which are obtained by free radical polymerization of vinyl acetate and N-vinyllactams in the presence of a polyether are preferred.

Corresponding copolymers are obtained by free radical polymerization of a mixture of
i) from 30 to 80% by weight of N-vinyllactam,
ii) from 10 to 50% by weight of vinyl acetate and
iii) from 10 to 50% by weight of a polyether,
   with the proviso that the sum of i), ii) and iii) is equal to 100% by weight.

According to an embodiment of the invention, preferred copolymers, obtainable from:
i) from 30 to 70% by weight of N-vinyllactam
ii) from 15 to 35% by weight of vinyl acetate and
iii) from 10 to 35% by weight of a polyether, are used.

Particularly preferably used copolymers are obtainable from:
i) from 40 to 60% by weight of N-vinyllactam
ii) from 15 to 35% by weight of vinyl acetate and
iii) from 10 to 30% by weight of a polyether.

Very particularly preferably used copolymers are obtainable from
i) from 50 to 60% by weight of N-vinyllactam
ii) from 25 to 35% by weight of vinyl acetate and
iii) from 10 to 20% by weight of a polyether.

The proviso that the sum of the components i), ii) and iii) is equal to 100% by weight is also true for the preferred and particularly preferred compositions.

N-vinylcaprolactam or N-vinylpyrrolidone or mixtures thereof are suitable as the N-vinyllactam. N-vinylcaprolactam is preferably used.

Accordingly, an amphiphilic copolymer of N-vinylcaprolactam, vinyl acetate and polyether is particularly preferred.

Polyethers serve as the grafting base. Suitable polyethers are preferably polyalkylene glycols. The polyalkylene glycols may have molecular weights of from 1000 to 100 000 Da [Dalton], preferably from 1500 to 35 000 Da, particularly preferably from 1500 to 10 000 Da. The molecular weights are determined starting from the OH number measured according to DIN 53240.

Particularly preferred polyalkylene glycols are polyethylene glycols. Furthermore, polypropylene glycols, polytetrahydrofurans or polybutylene glycols, which are obtained from 2-ethyloxirane or 2,3-dimethyloxirane, are also suitable.

Suitable polyethers are also random or block copolymers of polyalkylene glycols obtained from ethylene oxide, propylene oxide and butylene oxides, such as, for example, polyethylene glycol-polypropylene glycol block copolymers. The block copolymers may be of the AB or of ABA type.

The preferred polyalkylene glycols also include those which are alkylated at one terminal OH group or at both terminal OH groups. Suitable alkyl radicals are branched or straight-chain C₁- to C22-alkyl radicals, preferably C₁-C₁₈-alkyl radicals, for example methyl, ethyl, n-butyl, isobutyl, pentyl, hexyl, octyl, nonyl, decyl, dodecyl, tridecyl or octadecyl radicals.

General processes for the preparation of the copolymers used according to the invention are known per se. The preparation is effected by free radical polymerization, preferably in solution, in nonaqueous, organic solvents or in mixed nonaqeuous/aqueous solvents. Suitable preparation processes are described, for example, in WO 2007/051743 and WO 2009/013202, the disclosure of which with regard to the preparation process is hereby incorporated by reference.

According to an embodiment of the invention, the film-like dosage forms are obtained by melt extrusion. In the melt extrusion, all of the ingredients (active substance, polymer, additives) are melted together with the aid of a melt extruder and extruded via a slot die. After cooling, the resulting film can be cut into the suitable final size. A particular embodiment of the melt extrusion is designed so that extrusion is effected by means of a round or slot die and a calender having at least two rolls is loaded with the resulting extrudate. A homogeneous film leaves the calender. Usually, oral films are from 20 to 1000 μm, preferably 50-500 μm, thick. The active substance is present either finely suspended in crystalline or amorphous form or dissolved in the final film, the suspension representing by far the most frequent case.

According to a further embodiment of the invention, a suitable production method is evaporation. Here, the film-forming polymer, active substance and further additives are dissolved in a common solvent. Possible solvents are water or organic solvents, for example alcohols, such as ethanol, n-propanol, isopropanol, ketones, such as acetone, esters, such as ethyl acetate, butyl acetate, hydrocarbons, amides, such as dimethylacetamide, dimethylformamide. These solvents can be mixed with one another or with water in weight ratios which can be chosen according to requirements. Ethanol/water are preferred as a solvent.

The concentration of the solutions can be chosen freely within wide ranges and depends on the solubility of the components. In order to achieve sufficient film formation, however, preferably at least 1 to 40% by weight of film formers should be present.

The solution is usually mixed for a sufficient time and introduced into film molds (special rubber mats). In the next step, the solvent is removed. This is typically effected in a vacuum drying oven. The resulting films can then be removed from the molds and already have their final shape. This method of processing frequently manages without an additional cutting step.

The films can be also be drawn on Teflon sheets with a similar effort. However, the evaporation method can also be used in a continuous process. For this purpose, the polymer solution is applied to drying drums in a thin layer, dried by means of the energy of the drum and/or additional drying air and detached directly from the drum. This film must subsequently be cut into appropriate pieces. Instead of drum drying, the polymer solution can also be applied in a thin layer to a substrate sheet, which then passes through a heating tunnel for drying. Thereafter, the film with or without substrate sheet is cut into pieces.

In a particular embodiment, the films may be designed with a plurality of layers. As a result, incompatible components can be separated from one another and different active substance releases, higher adhesive power or different sensations of flavor can be achieved.

The films are usually packed either in multidose containers with up 100 films or individual packaging.

The use of the amphiphilic polymers for film production has substantial advantages over conventional film-forming polymers, owing to the ability to form solid solutions. Surprisingly, the amphiphilic polymer is completely neutral in taste and therefore also ideally suitable for aroma-free films.

The following additives can be added as further pharmaceutical excipients to the film in order to achieve certain properties:

Further polymers, active substances, plasticizers, colorants, antioxidants, emulsifiers, surfactants, stabilizers, preservatives, fillers, gel formers, sweeteners, acidifying agents, lubricants or aromas or mixtures thereof.

The use of microcrystalline cellulose can increase the decomposition rate. Further additives which can shorten the decomposition time are polyvinylpyrrolidones or vinylpyrrolidone copolymers, such as copovidone.

Further polymers which can be used are polyvinyl alcohols, polyvinyl alcohol-polyethylene glycol graft copolymer (commercially available as Kollicoat® IR, from BASF), polyethylene glycols, poloxamers, pullulan, starch and also modified starches, gelatin, hydroxyalkylated cellulose derivatives, carboxyalkylated cellulose derivatives or acrylic acid-methacrylic acid copolymers.

It is also possible to use mixtures of these polymers.

Furthermore, the films may also comprise disintegrants, such as crospovidone, croscarmellose, hydroxypropylcellulose having a low degree of substitution or crosslinked sodium carboxymethyl starch.

Mucoadhesive films, i.e. films which are intended for a relatively long residence time in the mouth, can likewise be produced. For this purpose, further polymers which have mucoadhesive properties are additionally incorporated. In particular, polycarbophil, polyacrylic acid, carrageenan, guar gum, alginates, xanthane, pectin, galactomannans, chitosan and also cellulose ethers are suitable here. These additional polymers can be used in amounts of from 1 to 95% by weight, preferably from 2 to 70% by weight.

For prolonging the duration of action, the incorporation of retard polymers may also be advisable. Ethylcellulose, ethyl acrylate-methyl methacrylate copolymer, ethyl acrylate-methyl methacrylate-trimethylammoniumethyl methacrylate copolymer, polyvinyl acetate, and ethylene-vinyl acetate copolymers are particularly suitable for this purpose.

In order to improve the organoleptic properties of the oral films, it is possible, as already mentioned, to add flavor improvers in the form of aromas or other sweeteners.

Additives such as cyclodextrins or resinates, which can be added to the polymers, are also flavor-masking. Surprisingly, in some cases the polymer alone is also sufficient for masking an unpleasant flavor of the active substance. This is possibly caused by the incorporation of the active substance in micelles of the amphiphilic polymer.

Saliva-influencing substances which are simultaneously also flavor-influencing can likewise be added, for example citric acid, tartaric acid, glucose, fructose, sucrose, mannitol, sorbitol, erythritol, isomalt, aspartame and saccharine. Typical concentrations are in the range of 1-20% by weight.

The addition of plasticizers (0-20% by weight) can improve the texture of the films, so that they are more readily extrudable or disintegrate more rapidly. Suitable additives here are in particular short- and medium-chain polyethylene glycols. It is also possible to use higher molecular polyethylene glycols. Furthermore, propylene glycols, glycerol and other polyols may be used. Surfactants too have particularly plasticizing properties with respect to polymers. The following may be mentioned in particular here: TPGS, polysorbate 20, 40, 60, 80, Span 20, stearic acid or salts thereof, glyceryl monostearate, sorbitan laurate, sodium laurylsulfate, docusate sodium, poloxamers, ethoxylated castor oil, hydrogenated ethoxylated castor oil, macrogol fatty alcohol ethers, macrogol fatty acid esters, macrogol sorbitan fatty alcohol ethers, macrogol sorbitan fatty acid esters, lecithin.

The abovementioned amounts in % by weight of additional pharmaceutical excipients are based on the total formulation.

Color-imparting agents or in particular pigments can likewise be added in order to impart a corresponding color to the film.

One or more active substances may be incorporated. Usually 1-50% by weight, preferably 2-30% by weight, of active substance are incorporated into the formulation, it also being possible for the amounts to differ therefrom, depending on the activity of the active substance. The film-like dosage forms according to the invention can in principle be used for all active substances. Particularly readily water-soluble as well as very sparingly water-soluble active substances can be used.

According to the invention, in particular active substances which are suitable for applications in transdermal dosage forms, such as, for example, hormones or opioid analgesics, or which are particularly frequently used in geriatrics or pediatrics are processed.

Benzodiazepines, antihypertensives, vitamins, cytostatics—in particular taxol, anesthetics, neuroleptics, antidepressants, antiviral agents, such as, for example, anti-HIV agents, antibiotics, antimycotics, antidementives, fungicides, chemotherapeutics, urologicals, platelet aggregation inhibitors, sulfonamides, spasmolytics, hormones, immunoglobulins, sera, thyroid therapeutics, psychopharmaceuticals, anti-Parkinson agents and other antihyperkinetics, ophthalmologicals, neuropathy preparations, calcium metabolism regulators, muscle relaxants, lipid-lowering agents, liver therapeutics, coronary agents, cardiac agents, immunotherapeutics, regulatory peptides and their inhibitors, hypnotics, sedatives, gynecologicals, anti-gout agents, fibrinolytics, enzyme preparations and transport proteins, enzyme inhibitors, emetics, perfusion promoters, diuretics, diagnostics, corticoids, cholinergics, biliary therapeutics, antiasthmatics, bronchospasmolytics, beta-receptor blockers, calcium antagonists, ACE inhibitors, arteriosclerosis agents, anti-inflammatory agents, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, weight-reducing agents may be mentioned as examples here.

In particular, the dosage forms according to the invention are suitable for the following active substances:

Nicotine, nitroglycerine and derivatives thereof, loperamide (antidiarrheal agent), flurazepam (anxiolytic agent), famotidine (antacid), dicyclomine (muscle relaxant), ketoprofen (cox inhibitor).

Antimicrobial substances, such as chlorhexidine gluconate, PVP-iodine, cetylpyridinium chloride, benzalkonium chloride, antibiotics.

Cortisones, such as hydrocortisone, betamethasone, dexamethasone.

Antihistamine agents, such as loratadine, desloratadine, cetirizine, acrivastine, diphenhydramine, diphenhydramine hydrochloride, azatidine maleate, chlorpherinamine, chlorpherinamine maleate, tiprolidine hydrochloride

Prazoles such as omeprazole, pantoprazole, lansoprazole. Triptans, such as zolmitriptan, sumatriptan succinate, almotriptan, eletriptan. Opioids, such as oxycodone.

The films obtained according to the invention can, as mentioned, be used as oral forms, in particular as forms rapidly decomposing in the oral cavity or in the pharynx, or as transdermal forms. Transdermal systems can be designed as matrix- or membrane-controlled forms. Here, the matrix systems may have a one-layer or multilayer structure, it being necessary for the layer resting on the skin to be tacky. This tack can be achieved by the use of known tacky polymers, such as polyisobutylene, acrylate-methacrylate polymers or silicone adhesives having very low glass transitions temperatures (less than 10° C.) or by the use of relatively large amounts of plasticizer. Transdermal systems usually have a backing layer and a release liner. The release liner is peeled off prior to application to the skin, and the backing layer forms the sealing layer of the system and ensures that the back is not tacky but has an attractive appearance and is occlusive.

The amphiphilic copolymers have substantial advantages over the prior art for these intended uses.

In the melt extrusion process, this class of compounds has advantages owing to their outstanding extrudibility because of their comparatively low glass transition temperature <100° C. Conventional polymers for film formation have glass transition temperature of >100° C. (for example hydroxypropylmethylcellulose/HPMC or hydroxy-propylcellulose/HPC, alginates, carrageenan). Because even particularly sparingly soluble active substances can be dissolved in the molecular state in the polymer, this invention permits access to a completely novel dosage form for many active substances.

In the evaporation process, also referred to as “film casting”, the amphiphilic polymer shows further strengths. Because even particularly sparingly soluble active substances are dissolved in the polymer, it is also not possible for sedimentation to occur during the drying phase in the polymer. Thus, active substance films comprising the amphiphilic polymer are distinguished in that they show particularly good content uniformity. With the use of conventional hydrophilic polymers for the evaporation method, the suspended active substance particles separate out so that inhomogeneous active substance distributions in the film result.

Oral films can be formulated in such a way that they disintegrate rapidly after being taken into the oral cavity. However, the films can also be formulated by means of special additives so that they are mucoadhesive and remain for a relatively long time in the oral cavity and release the active substance. In this way, sustained release of the active substance can be ensured.

### EXAMPLES

Films were produced by means of either melt extrusion or evaporation. The melt extrusion was effected in a two-screw extruder, screw diameter 16 mm, a length-to-diameter ratio of 40, and at 200 rpm screw speed. Extrusion was effected by means of a slot die having the dimensions 3 cm×0.5 mm. The film thickness can be adjusted by stretching the still soft film on a rotating belt or by 2 calender rolls which have an appropriate spacing.

A polyvinylcaprolactam-polyvinyl acetate-polyethylene glycol graft polymer commercially available under the name Soluplus® (from BASF) and having an average molecular weight Mw (determined by gel permeation chromatography) of from 90 000 to 140 000 g/mol, referred to below as “polymer”, was used as the film-forming amphiphilic copolymer.

General method for the production of the films

The products of the evaporation were produced using sufficient solvent (1:1 ethanol/water mixtures). The pulverulent substances were completely dissolved in the solvent with stirring. The liquid was poured into special rubber mats. Drying was effected in a vacuum drying oven for 5 h at 30° C. The resulting film was suitably cut.

For dissolution of the film, USP (US Pharmacopoeia) apparatus 2 (paddle method), 37° C., 900 ml of 0.08 N-HCl, 75 rpm (BTWS 600, Pharmatest) was used. For this purpose, the film was clamped in a slide frame (35×23 mm) and immersed in a release apparatus by means of a special apparatus. The orientation of the slide frame was radial and the distance to the liquid surface was 3 cm.

The time which was required until the film had the first hole (initial dissolution time) or had completely dissolved (complete dissolution time) was measured.

The film thickness was determined by means of a layer thickness measuring apparatus (Minitest 600BFN2). The elongation at break of the films was measured according to DIN 53504. The films were stored for 24 hours at 25° C. and 54% relative humidity before the measurement.

Abbreviation: DE water=demineralized water

### Example 1

1200 g of polymer and 300 g of famotidine (melting point 163° C.) were weighed into a Turbula mixing container and mixed for 10 minutes in the T1OB Turbula mixer.

The mixture was extruded under the following conditions:
zone temperature of 1st cylinder: 20° C.; 2nd cylinder: 40° C.
zone temperature from 3rd cylinder onward: 160° C.
screw speed 200 rpm
throughput: 300 g/h

The resulting film had a thickness of 80 μm and exhibited an elongation at break of 26%. The initial dissolution time in DE water was 20 seconds. The elongation at break is the percentage increase in length on tearing of film defined in the DIN standard.

### Example 2

1200 g of polymer, 10 g of docusate sodium and 300 g of loperamide (melting point 222° C.) were weighed into a Turbula mixing container and mixed for 10 minutes in the T10B Turbula mixer.

The mixture was extruded under the following conditions:
zone temperature of 1st cylinder: 20° C.; 2nd cylinder: 40° C.
zone temperature from 3rd cylinder onward: 180° C.
screw speed 200 rpm
throughput: 200 g/h

The resulting film had a thickness of 88 μm and exhibited an elongation at break of 22%. The initial dissolution time in DE water was 23 seconds.

### Example 3

1200 g of polymer and 300 g of cetirizine (melting point 115° C.) were weighed into a Turbula mixing container and mixed for 10 minutes in the T10B Turbula mixer.

The mixture was extruded under the following conditions:
zone temperature of 1st cylinder: 20° C.; 2nd cylinder: 40° C.
zone temperature from 3rd cylinder onward: 110° C.
screw speed 200 rpm
throughput: 400 g/h

The resulting film had a thickness of 79 μm and exhibited an elongation at break of 57%. The initial dissolution time in DE water was 21 seconds.

### Example 4

1000 g of polymer and 250 g of ketoprofen (melting point 94° C.) were weighed into a Turbula mixing container and mixed for 10 minutes in the T10B Turbula mixer.

The mixture was extruded under the following conditions:
zone temperature of 1st cylinder: 20° C.; 2nd cylinder: 40° C.
zone temperature from 3rd cylinder onward: 100° C.
screw speed 200 rpm
throughput: 600 g/h

The resulting film had a thickness of 119 μm and exhibited an elongation at break of 53%. The initial dissolution time in DE water was 34 seconds.

### Example 5

1100 g of polymer, 400 g of Kollidon VA 64, 100 g of PEG 1500 and 200 g of itraconazole (melting point 166° C.) were weighed into a Turbula mixing container and mixed for 10 minutes in the T1OB Turbula mixer.

The mixture was extruded under the following conditions:
zone temperature of 1st cylinder: 20° C.; 2nd cylinder: 40° C.
zone temperature from 3rd cylinder onward: 130° C.
screw speed 200 rpm
throughput: 300 g/h

The resulting film had a thickness of 94 μm and exhibited an elongation at break of 31%. The initial dissolution time in DE water was 120 seconds.

### Example 6

1000 g of polymer, 200 g of Kollidon 30, 100 g of polyethylene glycol 1500 and 200 g of naproxen (melting point 157° C.) were weighed into a Turbula mixing container and mixed for 10 minutes in the T10B Turbula mixer.

The mixture was extruded under the following conditions:
zone temperature of 1st cylinder: 20° C.; 2nd cylinder: 40° C.
zone temperature from 3rd cylinder onward: 160° C.
screw speed 200 rpm
throughput: 600 g/h

The resulting film had a thickness of 140 μm and exhibited an elongation at break of 24%. The initial dissolution time in DE water was 48 seconds.

### Example 7

1000 g of polymer, 100 g of Kollidon CL-M and 300 g of cinnarizine (melting point 122° C.) were weighed into a Turbula mixing container and mixed for 10 minutes in the T1OB Turbula mixer.

The mixture was extruded under the following conditions:
zone temperature of 1st cylinder: 20° C.; 2nd cylinder: 40° C.
zone temperature from 3rd cylinder onward: 130° C.
screw speed 200 rpm
throughput: 600 g/h

The resulting film had a thickness of 170 μm and exhibited an elongation at break of 25%. The initial dissolution time in DE water was 50 seconds.

### Example 8

1000 g of polymer and 10 g of cetylpyridinium chloride were extruded to give a film having a film thickness of 53 μm.

The mixture was extruded under the following conditions:
zone temperature of 1st cylinder: 20° C.; 2nd cylinder: 40° C.
zone temperature from 3rd cylinder onward: 130° C.
screw speed 200 rpm
throughput: 600g/h

The resulting film exhibited an elongation at break of 50%. The initial dissolution time in DE water was 15 seconds. 138 seconds were required for complete dissolution of the film.

### Example 9

1000 g of polymer and 50g of desloratadine were extruded without further additions to give a film having a film thickness of 170 μm.

The mixture was extruded under the following conditions:
zone temperature of 1st cylinder: 20° C.; 2nd cylinder: 40° C.
zone temperature from 3rd cylinder onward: 130° C.
screw speed 200 rpm
throughput: 600g/h

The resulting film exhibited an elongation at break of 43%. The initial dissolution time in DE water was 140 seconds. 990 seconds were required for complete dissolution of the film.

### Example 10

5 g of polymer and 2 g of felodipine were dissolved in 40 ml of ethanol and drawn to give a film. After drying, a thin film having a film thickness of 37 μm was obtained. The measured elongation at break of the film was 49%. The initial dissolution time in DE water was 10 seconds. 65 seconds were required for complete dissolution of the film.

### Example 11

4 g of polymer, 1.5 g of famotidine and 0.1 g of saccharine sodium were dissolved in 30 ml of ethanol and drawn to give a film. After drying, a thin film having a film thickness of 40 μm was obtained.

The measured elongation at break of the film was 46%. The initial dissolution time in DE water was 12 seconds. 69 seconds were required for complete dissolution of the film.

### Example 12

6 g of polymer and 2.2 g of cinnarizine were dissolved in 30 ml of isopropanol and drawn to give a film. After drying, a thin film having a film thickness of 52 μm was obtained.

The measured elongation at break of the film was 47%. The initial dissolution time in DE water was 11 seconds. 62 seconds were required for complete dissolution of the film.

### Example 13

6 g of polymer, 2 g of PEG 400 and 1.2 g of felodipine were dissolved in 30 ml of ethanol and drawn. After drying, a thin film having a film thickness of 57 μm was obtained.

The measured elongation at break of the film was 64%. The complete dissolution time in DE water was 10 seconds.

### Example 14

5 g of polymer, 1.5 g of PEG 1500, 0.1 g of aspartame and 1.0 g of loratadine were dissolved in 20 ml of isopropanol and 10 ml of dimethylacetamide and drawn. After drying, under reduced pressure a thin film having a film thickness of 44 μm was obtained.

The measured elongation at break of the film was 51%. The initial dissolution time in DE water was 10 seconds. 71 seconds were required for complete dissolution of the film.

Example 15

3 g of polymer, 2 g of HPMC, 1.0 g of triethyl citrate, 0.1 g of riboflavin and 2.2 g of famotidine were dissolved in 30 ml of ethanol and drawn to give a film. After drying, a thin film having a film thickness of 48 μm was obtained.

The measured elongation at break of the film was 37%. The initial dissolution time in DE water was 11 seconds. The complete dissolution in DE water took place after 70 seconds.

### Example 16

3 g of polymer, 2 g of HPC, 0.1 g of tartaric acid and 2 g of loperamide were dissolved in 20 ml of ethanol and 10 ml of dimethylformamide and drawn to give a film. After drying, a thin film having a film thickness in the range of 42 μm was obtained. The measured elongation at break of the film was 39%. The initial dissolution time in DE water was 10 seconds. The complete dissolution in DE water took place after 69 seconds.

### Example 17

2 g of polymer, 2 g of polyvinyl alcohol-polyethylene glycol graft copolymer (Kollicoat IR) and 0.5 g of chlorhexidine gluconate were dissolved in 20 ml of water and drawn to give a film. After drying, a thin film having a film thickness of 45 μm was obtained.

The measured elongation at break of the film was 71%. The initial dissolution time in DE water was 9 seconds. The complete dissolution in DE water took place after 51 seconds.

1. A film-like pharmaceutical dosage form comprising one or more amphiphilic copolymers as film formers and one or more active substances.

2. The dosage form according to claim 1, wherein the amphiphilic copolymers comprise copolymers of polyethers, N-vinyl monomers and further vinyl monomers.

3. The dosage form according to claim 1 wherein the amphiphilic copolymers are obtained by free radical polymerization of vinyl acetate and N-vinyllactams in the presence of a polyether.

4. The dosage form according to claim 1 wherein the amphiphilic copolymers are obtained by free radical polymerization of a mixture of i) from 30 to 80% by weight of N-vinyllactam, ii) from 10 to 50% by weight of vinyl acetate and iii) from 10 to 50% by weight of a polyether, with the proviso that the sum of i), ii) and iii) is equal to 100% by weight.

5. The dosage form according to claim 1 wherein the amphiphilic copolymer comprises a copolymer of N-vinylcaprolactam, vinyl acetate and a polyether.

6. The dosage form according to claim 1, wherein the dosage form comprises amphiphilic copolymers in amounts of from 1 to 100% by weight, based on the total amount of pharmaceutical excipients.

7. The dosage form according to claim 6, wherein the dosage form comprises amphiphilic copolymers in amounts of from 10 to 90% by weight, based on the total amount of pharmaceutical excipients.

8. The dosage form according to claim 7, wherein the dosage form comprises amphiphilic copolymers in amounts of from 40 to 70% by weight, based on the total amount of pharmaceutical excipients.

9. The dosage form according to claim 1, wherein the dosage form comprises further pharmaceutical excipients selected from further polymers, active substances, plasticizers, colorants, antioxidants, emulsifiers, surfactants, stabilizers, preservatives, fillers, gel formers, sweeteners, acidifying agents, lubricants or aromas or mixtures thereof.

10. The dosage form according to claim 9, wherein the dosage form comprises further polymers selected from povidone, copovidone, polyvinyl alcohols, polyvinyl alcohol-polyethylene glycol graft copolymers, polyethylene glycols, poloxamers, pullulan, starch, modified starches, gelatin, hydroxyalkylated cellulose derivatives, carboxyalkylated cellulose derivatives, acrylic acid-methacrylic acid copolymers or mixtures thereof.

11. The dosage form according to claim 1, further comprising disintegrants.

12. The dosage form according to claim 1, further comprising mucoadhesive polymers selected from the group consisting of polycarbophil, polyacrylic acid, carrageenan, guar gum, alginates, galactomannans, pectins, chitosan and cellulose ethers.

13. The dosage form according to claim 1, wherein the dosage form comprises from 0.1 to 50% by weight, based on the total formulation, of active substance.

14. A process for the preparation of film-like dosage forms, the process comprising mixing one or more amphiphilic copolymers as film formers with one or more active substances to provide a mixture, and shaping the mixture into a film.

15. The process according to claim 14, wherein shaping the mixture comprises melt extrusion.

16. The process according to claim 14, wherein the one or more film formers and the one or more active substances are mixed in a solvent to provide a solution, the solution is drawn to give a film and a film forms as a result of evaporation of the solvent.

17. The process according to claim 16, wherein water, alcohols, ketones, esters, hydrocarbons, amides or mixtures thereof are used as the solvent.

18. The dosage form according to claim 1, wherein the film has a thickness in the range from 37 μm to 170 μm.

19. The dosage form according to claim 1, wherein the copolymer has an average molecular weight in the range from 90,000 to 140,000 g/mol.

20. The process according to claim 14, wherein the film has a thickness in the range from 37 μm to 170 μm.
